(19) United States
(12) Patent Application Publication
(10) Pub. No.: US 2009/0209612 A1
(43) Pub. Date: Aug. 20, 2009

(54) PROCESS FOR THE PREPARATION OF ATORVASTATIN

(76) Inventors: Jitendra A. Sattigeri, Gurgaon (IN); Mohammad Salman, Princeton, NJ (US); Shobhana Rawat, Uttar Pradesh (IN); Sachin Sethi, Yamuna Nagar (IN)

Correspondence Address: RANBAXY INC.;INTELLECTUAL PROPERTY DEPT., 600 COLLEGE ROAD EAST, SUITE 2100, PRINCETON, NJ, 08540, US

(21) Appl. No.: 11/569,843

(22) PCT Filed: May 31, 2005
(86) PCT No.: PCT/IB05/01526

§ 371 Date: Sep. 8, 2008

(30) Foreign Application Priority Data
May 31, 2004 (IN) 990/DEL/2004

(51) Int. Cl. A61K 31/40; C07D 207/00

(52) U.S. Cl. 514/423; 548/537

(57) ABSTRACT

The invention relates to processes for the preparation of atorvastatin. Atorvastatin is known by the chemical name [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid. The hemi-calcium salt of atorvastatin is useful as an inhibitor of the enzyme 3-hydroxy-3-methylglutaryl-coenzyme A reductase (HMG-CoA reductase). The invention also relates to pharmaceutical compositions that include the atorvastatin or a pharmaceutically acceptable salt thereof and to use of said compositions for treating hypolipidemia, hypocholesterolemia and atherosclerosis.

### FIELD OF THE INVENTION

The field of the invention relates to processes for the preparation of atorvastatin. Atorvastatin is known by the chemical name [R—(R*, R*)]-2-(4-fluorophenyl)-β,δ-dihydroxy-5-(1-methylethyl)-3-phenyl-4-[(phenylamino)carbonyl]-1H-pyrrole-1-heptanoic acid. The hemi-calcium salt of atorvastatin is useful as an inhibitor of the enzyme 3-hydroxy-3-methylglutaryl-coenzyme A reductase (HMG-CoA reductase). The invention also relates to pharmaceutical compositions that include the atorvastatin or a pharmaceutically acceptable salt thereof and to use of said compositions for treating hypolipidemia, hypocholesterolemia and atherosclerosis.

### BACKGROUND OF THE INVENTION

Cardiovascular disease and its associated maladies, dysfunctions and complications are a principal cause of disability and the chief cause of death. One specific factor significantly contributing to this pathophysiologic process is atherosclerosis, which has been generally recognized as the leading health care problem both with respect to mortality and health care costs.

Atherosclerosis is characterized by the deposition of fatty substances, primarily cholesterol, resulting in plaque formation on the inner surface of the arterial wall and degenerative change within it.

It is now well established that vascular blockage and cardiovascular disorders including myocardial infarction, coronary heart disease, hypertension and hypotension, cerebrovascular disorders including stroke, cerebral thrombosis and memory loss due to stroke; peripheral vascular disease and intestinal infarction are caused by blockage of arteries and arterioles by atherosclerotic plaque. Atherosclerotic plaque formation is multi-factorial in its production. Hypercholesterolemia, especially elevated level of low-density lipoprotein cholesterol (LDL) is an important risk factor for atherosclerosis and arteriosclerosis and associated diseases.

The HMG-CoA reductase inhibitors (statins) have been used in reducing blood levels of LDL cholesterol level. Cholesterol is produced via the mevalonic acid pathway. Reducing the formation of mevalonic acid, a precursor to cholesterol, leads to a corresponding decrease in hepatic cholesterol biosynthesis with a reduction in the cellular pool of cholesterol.

The disclosures of U.S. Pat. Nos. 4,681,893 5,216,174; 5,097,045; 5,103,024; 5,124,482; 5,149,837; 5,155,251; 5,245,047; 5,273,995; 5,248,793 and 5,397,792 are relevant to atorvastatin, and these disclosures are incorporated herein by reference. WO 02/057274 discloses a process for the synthesis of atorvastatin form V and phenylboronates as intermediate compounds, and also is incorporated herein by reference.

### SUMMARY OF THE INVENTION

In one embodiment, a process for the preparation of [R—(R*, R*)]-2-(4-fluorophenyl)-β,δ-dihydroxy-5-(1-methylethyl)-3-phenyl-4-[(phenylamino)carbonyl]-1H-pyrrole-1-heptanoic acid hemi calcium salt of Formula I, or other pharmaceutically acceptable salts thereof, is provided.

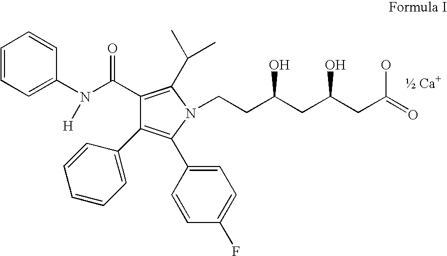

[R—(R*, R*)]-2-(4-fluorophenyl)-β,δ-dihydroxy-5-(1-methylethyl)-3-phenyl-4-[(phenylamino)carbonyl]-1H-pyrrole-1-heptanoic acid hemi calcium can be prepared by following illustrative reaction sequences as depicted below in Scheme I.

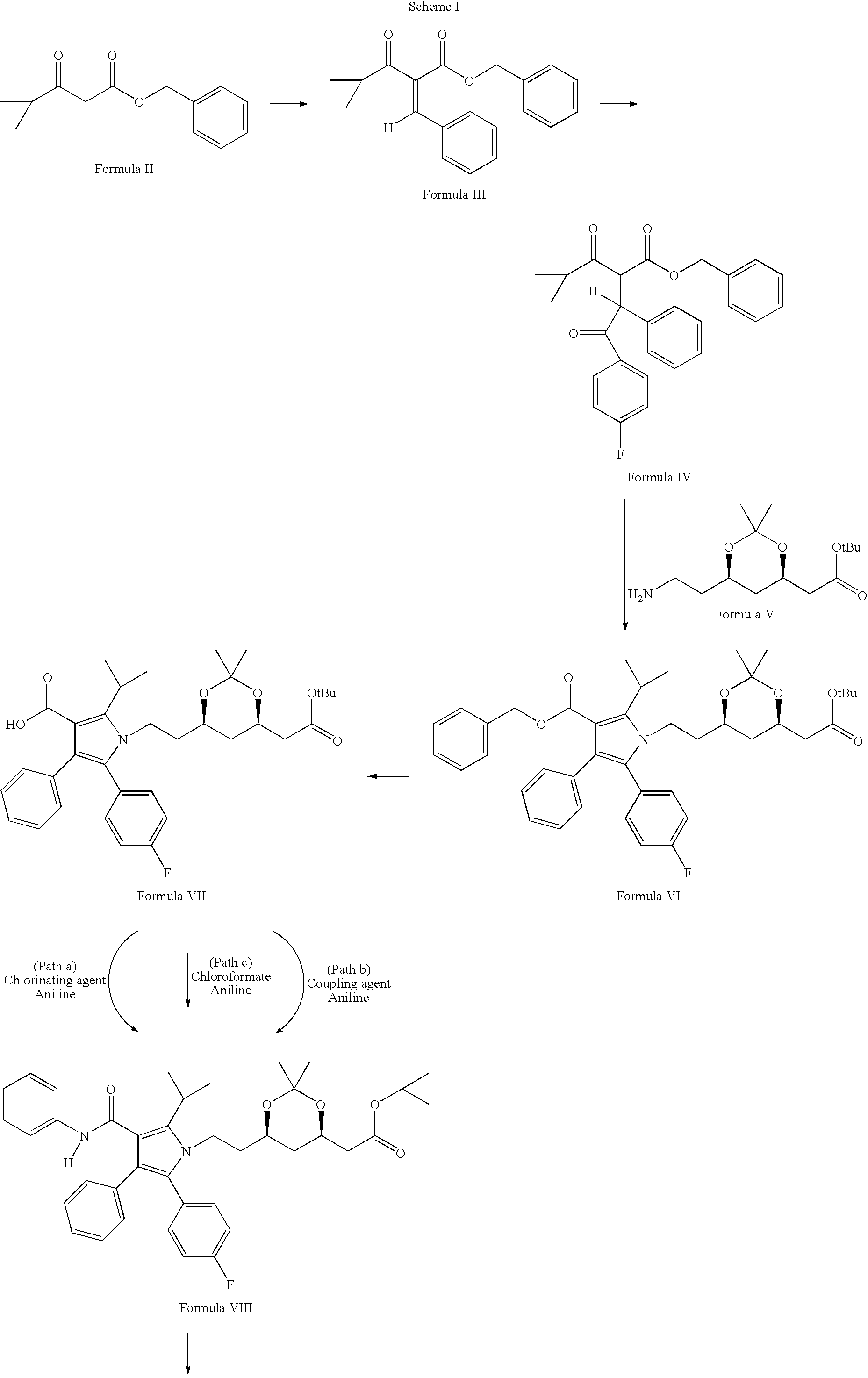

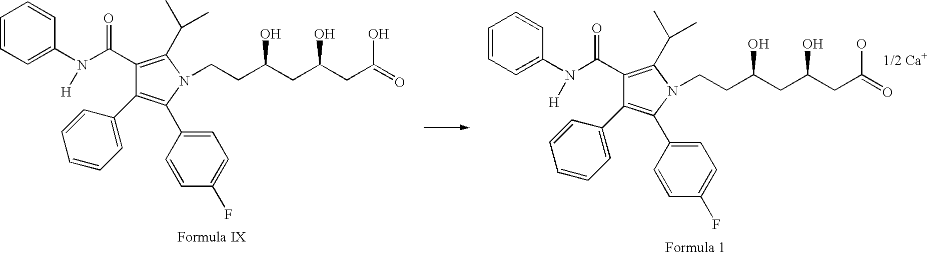

The process includes

(a) reacting 4-methyl-3-oxo-pentanoic acid benzyl ester of Formula II (as shown in Scheme I) with benzaldehyde to give 4-methyl-3-oxo-2-[1-phenyl-methylidene]-pentanoic acid benzyl ester of Formula III;

(b) reacting a compound of Formula III with 4-fluorobenzaldehyde to give 2-[2-(4-fluorophenyl)-2-oxo-1-phenylethyl]-4-methyl-3-oxo-pentanoic acid benzyl ester of Formula IV;

(c) reacting a compound of Formula IV with tert-butyl [(4R,6R)-6-(2-aminoethyl)-2,2-dimethyl-1,3-dioxan-4-yl]acetate of Formula V to give 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid benzyl ester of Formula VI;

(d) debenzylating 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid benzyl ester of Formula VI to give 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII;

(e)
1. converting 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII to corresponding acid chloride followed by reaction with aniline (Path a),
2. reacting 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII with aniline in the presence of a coupling agent (Path b), or
3. converting 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII to its corresponding mixed anhydride followed by reaction with aniline (Path c),
   to give (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII;

(f) hydrolyzing (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII to give a compound of Formula IX; and

(g) converting the compound of Formula IX into a hemi calcium salt of Formula I, or other pharmaceutically acceptable salts thereof.

In another embodiment, a pharmaceutical composition is provided that includes an effective amount of atorvastatin or a pharmaceutically acceptable salt thereof; and one or more pharmaceutically acceptable carriers, excipients or diluents.

In another embodiment, a method is provided for treating hypolipidemia, hypocholesterolemia and atherosclerosis in a warm-blooded animal, the method comprising providing a pharmaceutical composition to the warm-blooded animal that includes the atorvastatin or a pharmaceutically acceptable salt thereof.

The details of one or more embodiments of the inventions are set forth in the description below. Other features, objects and advantages of the inventions will be apparent from the description and claims.

### DETAILED DESCRIPTION OF THE INVENTION

[R—(R*, R*)]-2-(4-fluorophenyl)-β,δ-dihydroxy-5-(1-methylethyl)-3-phenyl-4-[(phenylamino)carbonyl]-1H-pyrrole-1-heptanoic acid hemi calcium of Formula I can be prepared according to scheme I. Thus, reacting 4-methyl-3-oxo-pentanoic acid benzyl ester of Formula II with benzaldehyde to give 4-methyl-3-oxo-2-[1-phenyl-methylidene]-pentanoic acid benzyl ester of Formula III, which on reaction with 4-fluorobenzaldehyde gives 2-[2-(4-fluorophenyl)-2-oxo-1-phenylethyl]-4-methyl-3-oxo-pentanoic acid benzyl ester of Formula IV, which on reaction with tert-butyl [(4R,6R)-6-(2-aminoethyl)-2,2-dimethyl-1,3-dioxan-4-yl]acetate of Formula V gives 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid benzyl ester of Formula VI, which on debenzylation gives 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII, which on

(a) conversion to corresponding acid chloride followed by reaction with aniline (Path a),

(b) reaction with aniline in the presence of a coupling agent (Path b), or

(c) conversion to corresponding mixed anhydride followed by reaction with aniline (Path c),

gives (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII, which on hydrolysis gives compound of Formula IX, which can be further converted to hemi calcium salt of Formula I, or other pharmaceutically acceptable salts of Formula IX by any of the methods known in the art.

The reaction of 4-methyl-3-oxo-pentanoic acid benzyl ester of Formula II with benzaldehyde to give 4-methyl-3-oxo-2-[1-phenyl-methylidene]-pentanoic acid benzyl ester of Formula III can be carried out in one or more solvents, including for example, xylene, toluene, heptane, hexane, dichloromethane, and mixtures thereof, in the presence of one or more organic bases, including for example, triethylamine, pyridine, piperidine, and β-alanine, and one or both organic acids, for example, glacial acetic acid and benzoic acid.

The reaction of 4-methyl-3-oxo-2-[1-phenyl-methylidene]-pentanoic acid benzyl ester of Formula III with 4-fluorobenzaldehyde can be carried out in one or more solvents, including for example, methanol, ethanol, propanol, and isopropanol, in the presence of one or more organic bases, including for example, triethylamine, and pyridine, and one or both catalysts, for example, sodium cyanide, 3-ethyl-5-(2-hydroxyethyl)-4-methyl thiazolium bromide and 3-benzyl-5-(2-hydroxyethyl)-4-methyl thiazolium chloride.

The reaction of 2-[2-(4-fluorophenyl)-2-oxo-1-phenylethyl]-4-methyl-3-oxo-pentanoic acid benzyl ester of Formula IV with tert-butyl [(4R,6R)-6-(2-aminoethyl)-2,2-dimethyl-1,3-dioxan-4-yl]acetate of Formula V can be carried out in one or more solvents, including for example, hexane, heptane, toluene, tetrahydrofuran, and a mixture thereof in various combinations in the presence of an acid, for example, pivalic acid or p-toluene sulfonic acid.

The debenzylation of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid benzyl ester of Formula VI can be carried out in one or more solvents, including for example, methanol, ethanol, propanol, dioxane, and a mixture thereof in the presence of a catalyst, for example, palladium on carbon and hydrogen.

The conversion of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII to its corresponding acid chloride (Path a) can be carried out with any chlorinating agent, including for example, oxalyl chloride, phosphorus pentachloride, and sulfonyl chloride in a solvent, for example, benzene, dichloromethane, tetrahydrofuran, toluene and xylene. The reaction of acid chloride with aniline to give (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII can be carried out in a solvent, including for example, benzene and toluene, and in the presence of an organic base, for example, triethylamine and pyridine.

The reaction of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII with aniline can be carried out in the presence of a coupling agent, including for example, O-benzotriazol-1-yl-N,N,N′,N′-tetramethyl uronium hexafluorophosphate (HBTU), bis(2-oxo-3-oxazolidinyl)phosphine (BOP), 1,3-dicyclohexycarbodiimide (DCC), 2-(1H-benzotriazole-1-yl)-1,1,3,3-tetramethyluronium tetrafluoroborate (TBTU), benzotriazole-1-yl-oxy-tris-pyrrolidino-phosphonium hexafluorophosphate (PyBOP) and carbonyldiimidazole (CDI) (Path b) in a solvent, for example, dimethylformamide, and a base, for example, diisopropylethylamine. The coupling reaction can also be achieved following any method known in the art.

The conversion of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII to a mixed anhydride can be carried out with any chloroformate, for example, isobutylchloroformate (path c) in the presence of a base, for example, triethylamine or pyridine in one or more solvents, including for example, tetrahydrofuran, toluene and dichloromethane. The reaction of mixed anhydride with aniline to give (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII can be carried out in the presence of p-toluene sulfonic acid in one or more solvents, for example, tetrahydrofuran, toluene and dichloromethane.

The conversion of (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII to 7-[2-(4-fluorophenyl)-5-isopropyl-3-phenyl-4-phenylcarbamoyl-pyrrole-1-yl]-3,5-dihydroxy-heptanoic acid of Formula I×can be carried out in a two step manner involving an initial acid catalyzed cleavage of ketal followed by base catalyzed hydrolysis of tert-butyl ester. The acid can be a mineral acid, for example, hydrochloric acid. The cleavage of ketal can be carried out by any cleavage method known in the art. The base can be, for example, lithium hydroxide, sodium hydroxide or potassium hydroxide.

[R—(R*, R*)]-2-(4-fluorophenyl)-β,δ-dihydroxy-5-(1-methylethyl)-3-phenyl-4-[(phenylamino)carbonyl]-1H-pyrrole-1-heptanoic acid of Formula IX can be converted into its corresponding hemi calcium salt of Formula I by any of the methods known in the art including those described in U.S. Pat. Nos. 5,273,995; 5,969,156; and 6,087,511.

In the above synthetic method where specific solvents, acids, bases, catalysts, etc., are mentioned, it is to be understood that the other solvent, acids, bases, catalysts, etc., may be used. Similarly, the reaction temperature and duration of reaction can be adjusted.

The resulting atorvastatin hemi-calcium salt may be formulated into ordinary dosage forms such as, for example, tablets, capsules, pills, solutions, etc. In these cases, the medicaments can be prepared by conventional methods with conventional pharmaceutical excipients.

The atorvastatin hemi-calcium can be administered for the prevention and treatment of hypolipidemia, hypocholesterolemia and atherosclerosis in a warm-blooded animal.

For the purpose of this disclosure, a warm-blooded animal is a member of the animal kingdom possessed of a homeostatic mechanism and includes mammals and birds. The salt is generally administered as part of a pharmaceutical composition with a pharmaceutically acceptable carrier, diluent or excipient and optionally other therapeutic ingredients. The salt may be conventionally formulated into tablets, capsules, suspensions, dispersions, injectables and other pharmaceutical forms. Any suitable route of administration may be employed, such as, for example, peroral or parenteral.

The present invention is further illustrated by the following examples which are provided merely to be exemplary of the invention and are not intended to limit the scope of the invention. Certain modifications and equivalents will be apparent to those skilled in the art and are intended to be included within the scope of the present invention.

### Example 1

### Preparation of 4-methyl-3-oxo-2-[1-phenyl-meth-(Z)-ylidene]-pentanoic acid benzyl ester of Formula III

To a solution of 4-methyl-3-oxo-pentanoic acid benzyl ester of Formula II (4.5 mmoles) in toluene (15 ml), benzaldehyde (4.9 mmoles), piperidine (0.02 ml) and acetic acid (0.054 ml) were added. The mixture was heated at reflux with azeotropic removal of water for about 4 to 6 hours. The reaction mixture was concentrated and residue extracted in dichloromethane. Organic layer was washed with 1N hydrochloric acid solution, sodium bicarbonate solution, brine. The organic layer was dried over anhydrous sodium sulphate and concentrated. The crude product was purified on column (silica gel, 100-200 mesh, 2% EtOAc-hexane).

### Example 2

### Preparation of 2-[2-(4-fluorophenyl)-2-oxo-1-phenylethyl]-4-methyl-3-oxo-pentanoic acid benzyl ester of Formula IV

4-methyl-3-oxo-2-[1-phenyl-methylidene]-pentanoic acid benzyl ester of Formula III (6.49 mmoles), 4-fluorobenzaldehyde (7.14 mmoles), 3-ethyl-5-(2-hydroxyethyl)-4-methyl thiazolium bromide (1.298 mmoles), triethylamine (6.49 mmoles) and ethanol (0.6 ml) were placed in a 30 ml vial, flushed with argon and the vial properly sealed. The reaction mixture was stirred at 70° C. for about 12 to 15 hours. To the reaction mixture, ethyl acetate was added. It was washed with water, 6N hydrochloric acid, again with water and brine, dried over anhydrous sodium sulphate, and concentrated to give crude product. The crude product was purified on column (silica gel 100-200 mesh) using 7% ethyl acetate in hexane.

### Example 3

### Preparation of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid benzyl ester of Formula VI

To a solution of 2-[2-(4-fluorophenyl)-2-oxo-1-phenylethyl]-4-methyl-3-oxo-pentanoic acid benzyl ester Formula IV (4.62 mmles) in heptane:toluene:tetrahydrofuran (4:1:1), tert-butyl [(4R,6R)-6-(2-aminoethyl)-2,2-dimethyl-1,3-dioxan-4-yl]acetate of Formula V (6.99 mmoles) and pivalic acid (4.768 mmoles) were added. The mixture was refluxed with azeotropic removal of water for about 22 to 25 hours. The reaction mixture was concentrated and ethyl acetate was added. It was washed with sodium bicarbonate solution and brine, dried over anhydrous sodium sulphate and concentrated to give crude product. The crude product was purified on column (silica gel, 100-200 mesh) using 7% ethyl acetate in hexane.

### Example 4

### Preparation of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII

To a solution of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid benzyl ester of Formula VI (0.8 g) in methanol:dioxan (2:8) mixture, 10% palladium carbon (50% wet, 60% w/w) was added. The resulting reaction mixture was hydrogenated at 40 psi for about 2.5 hours. After the reaction was over, the reaction mixture was passed through celite and the resulting solution was concentrated under vacuum to give the required product, which was further used as such for next step.

### Example 5

### Preparation of (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII

Path a: To a solution of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII (1 equiv) in benzene at 0° C. under argon, oxalyl chloride (2.0 equiv) was added drop wise. After the evolution of gas had ceased, the reaction mixture was heated on an oil bath at 70° C. for about 2 hours. The reaction mixture was evaporated to dryness to leave a residue. The residue was dissolved in benzene (dry) and added at ambient temperature to a solution of aniline (1.1 equiv.) in benzene. The reaction mixture was then heated at 70° C. till completion of reaction. Volatiles were removed in vacuo & the residue was purified on column (silica gel, 100-200 mesh).

Path b: To a solution of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII (1.2 mmole) in dimethylformamide (2.5 ml), diisopropylethylamine (2.4 mmole) and O-benzotriazol-1-yl-N,N,N′,N′-tetramethyl uronium hexafluorophosphate (HBTU) (1.2 mmoles) were added. To the resulting clear solution, aniline (1.2 mmoles) in dimethylformamide (0.5 ml) was added, and the reaction mixture was stirred at 50° C. to 60° C. overnight. To the reaction mixture, water was added and it was extracted with dichloromethane. The organic layer was separated and washed with water and brine. It was dried over anhydrous sodium sulphate and concentrated to get the crude product. The crude product was purified by column chromatography (silica gel, 100-200 mesh) using 10% ethyl acetate in hexane.

Path c: To a cooled solution of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII (2.6 mmole) in tetrahydrofuran (15 ml), triethylamine (2.6 mmole) followed isobutylchloroformate (2.6 mmoles) were added at about −15° C. The reaction mixture was stirred for about 15 minutes and then aniline (2.6 mmoles, in tetrahydrofuran) was added followed by addition of p-toluene sulfonic acid (0.26 mmoles). The reaction mixture was heated at about 55-60° C. overnight. To the reaction mixture, water was added and it was extracted with dichloromethane. The organic layer was separated, washed with 0.1N HCl, brine, dried over anhydrous sodium sulphate and concentrated to get the crude product. The crude product was purified by column chromatography (silica gel, 100-200 mesh) using 10% ethyl acetate in hexane.

### Example 6

### Preparation of [R—(R*. R*)]-2-(4-fluorophenyl)-β,δ-dihydroxy-5-(1-methylethyl)-3-phenyl-4-[(phenylamino)carbonyl]-1H-pyrrole-1-heptanoic acid hemi-calcium salt of Formula I

(a) To a solution of (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII in methanol and tetrahydrofuran (1:1), 1 N hydrochloric acid (3 equiv) was added and the mixture was stirred at an ambient temperature. After the complete hydrolysis of ketal, the reaction mixture was cooled to 0° C. and sodium hydroxide pellets (6 equiv) were added. The reaction was then allowed to stir at an ambient temperature. At the end of ester hydrolysis, solvents were removed and residue so obtained was dissolved in water; the aqueous layer was washed with ether and neutralized with 1 N hydrochloric acid. Organics were extracted into ethyl acetate, and concentrated to give a residue. The residue was then purified on column (silica gel 100-200 mesh) to give [R—(R*, R*)]-2-(4-fluorophenyl)-β,δ-dihydroxy-5-(1-methylethyl)-3-phenyl-4-[(phenylamino)carbonyl]-1H-pyrrole-1-heptanoic acid of Formula IX.

(b) To an aqueous solution of sodium salt of acid of Formula IX (prepared by adding 1 equivalent 1N sodium hydroxide solution), an aqueous solution (1M) of calcium acetate (0.55 equiv) was added drop wise. A white precipitate was obtained which was filtered off and washed with a copious amount of water, and dried in vacuum.

While the present invention has been described in terms of its specific embodiments, certain modifications and equivalents will be apparent to those skilled in the art and are included within the scope of the present invention.

We claim:

1. A process for the preparation of [R—(R*, R*)]-2-(4-fluorophenyl)-β,δ-dihydroxy-5-(1-methylethyl)-3-phenyl-4-[(phenylamino)carbonyl]-1H-pyrrole-1-heptanoic acid hemi calcium salt of Formula I, or other pharmaceutically acceptable salts thereof, 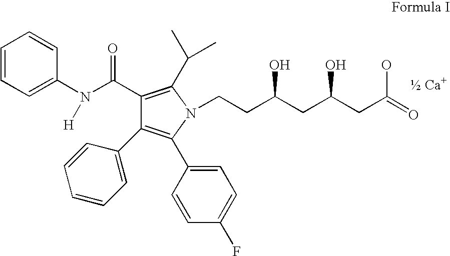
the process comprising:
(a) reacting 4-methyl-3-oxo-pentanoic acid benzyl ester of Formula II with benzaldehyde 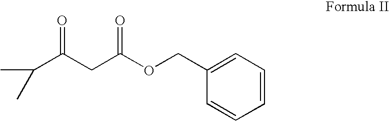
to give 4-methyl-3-oxo-2-[1-phenyl-methylidene]-pentanoic acid benzyl ester of Formula III; 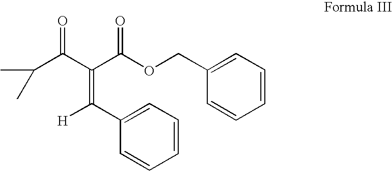
(b) reacting a compound of Formula III with 4-fluorobenzaldehyde to give 2-[2-(4-fluorophenyl)-2-oxo-1-phenylethyl]-4-methyl-3-oxo-pentanoic acid benzyl ester of Formula IV; 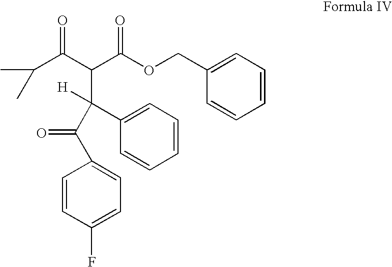
(c) reacting a compound of Formula IV with tert-butyl [(4R,6R)-6-(2-aminoethyl)-2,2-dimethyl-1,3-dioxan-4-yl]acetate of Formula V 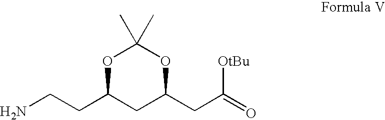
to give 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid benzyl ester of Formula VI; 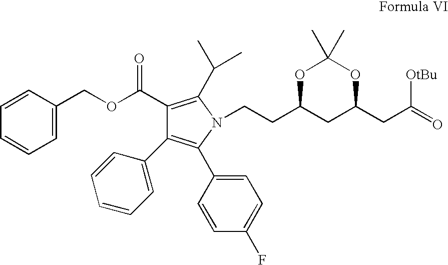
(d) debenzylating 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid benzyl ester of Formula VI to give 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII; 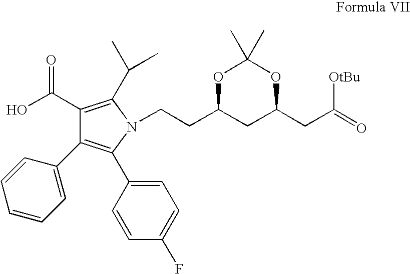
(e) converting 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII to give (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII; 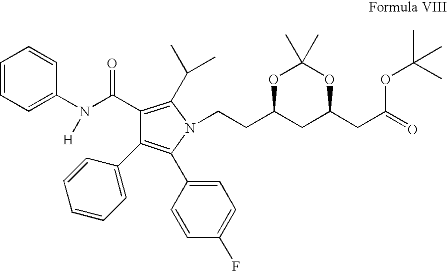
(f) hydrolyzing (6-{2-[2-(4-fluorophenyl)-5-isopropyl-3-phenyl-4 phenylcarbamoyl-pyrrole-1-yl]-ethyl}-2,2-dimethyl-[1,3]dioxan-4-yl)-acetic acid tert-butyl ester of Formula VIII to give a compound of Formula IX; and 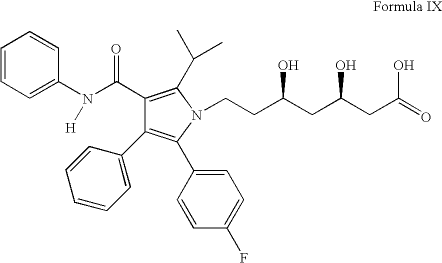
(g) converting the compound of Formula IX into a hemi calcium salt of Formula I, or other pharmaceutically acceptable salts thereof.

2. The process of claim 1, wherein the reaction of 4-methyl-3-oxo-pentanoic acid benzyl ester of Formula II with benzaldehyde to give 4-methyl-3-oxo-2-[1-phenyl-methylidene]-pentanoic acid benzyl ester of Formula III is carried out in the presence of an organic base.

3. The process of claim 2, wherein the organic base comprises one or more of triethylamine, pyridine, piperidine and β-alanine.

4. The process of claim 2 further comprising carrying out the reaction in the presence of an organic acid.

5. The process of claim 4, wherein the organic acid comprises one or both of acetic acid and benzoic acid.

6. The process of claim 1, wherein the reaction of 4-methyl-3-oxo-2-[1-phenyl-methylidene]-pentanoic acid benzyl ester of Formula III with 4-fluorobenzaldehyde to give 2-[2-(4-fluorophenyl)-2-oxo-1-phenylethyl]-4-methyl-3-oxo-pentanoic acid benzyl ester of Formula IV is carried out in the presence of a catalyst.

7. The process of claim 6, wherein the catalyst comprises one or both of 3-ethyl-5-(2-hydroxyethyl)-4-methyl thiazolium bromide and 3-benzyl-5-(2-hydroxyethyl)-4-methyl thiazolium chloride.

8. The process of claim 6, further comprising carrying out the reaction in the presence of an organic base.

9. The process of claim 8, wherein the organic base comprises one or both of triethylamine and pyridine.

10. The process of claim 1, wherein the reaction of 2-[2-(4-fluorophenyl)-2-oxo-1-phenylethyl]-4-methyl-3-oxo-pentanoic acid benzyl ester of Formula IV with tert-butyl [(4R,6R)-6-(2-aminoethyl)-2,2-dimethyl-1,3-dioxan-4-yl]acetate of Formula V to give 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid benzyl ester of Formula VI is carried out in the presence of an acid.

11. The process of claim 10, wherein the acid comprises one or both of pivalic acid and p-toluene sulfonic acid.

12. The process of claim 1, wherein the debenzylation of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid benzyl ester of Formula VI to give 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VI is carried out in the presence of a catalyst and hydrogen.

13. The process of claim 12, wherein the catalyst is palladium on carbon.

14. The process of claim 1 wherein the conversion of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of formula VII to (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII is effected through conversion of Formula VII to its corresponding acid chloride followed by its reaction with aniline.

15. The process of claim 14, wherein the acid chloride of Formula VII is prepared by reaction of Formula VII with a chlorinating agent.

16. The process of claim 15, wherein the chlorinating agent comprises one or more of oxalyl chloride, phosphorus pentachloride, and sulfonyl chloride.

17. The process of claim 14, wherein the reaction of acid chloride of Formula VII with aniline to give (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII is carried out in the presence of an organic base.

18. The process of claim 1, wherein the conversion of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII to (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII is effected through reaction of Formula VII with aniline in the presence of a coupling agent.

19. The process of claim 18, wherein the coupling agent comprises one or more of O-benzotriazol-1-yl-N,N,N′,N′-tetramethyl uronium hexafluorophosphate, bis(2-oxo-3-oxazolidinyl)phosphine, 1,3-dicyclohexycarbodiimide, 2-(1H-benzotriazole-1-yl)-1,1,3,3-tetramethyluronium tetrafluoroborate, benzotriazole-1-yl-oxy-tris-pyrrolidino-phosphonium hexafluorophosphate and carbonyldiimidazole.

20. The process of claim 1, wherein the conversion of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII to (6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII is effected through conversion of Formula VII to its corresponding mixed anhydride followed by reaction with aniline.

21. The process of claim 20, wherein the mixed anhydride of Formula VII is prepared by reaction of Formula VII with a chloroformate in the presence of a base.

22. The process of claim 20, wherein the reaction of the mixed anhydride of 1-[2-(6-tert-butoxycarbonylmethyl-2,2-dimethyl-[1,3]dioxan-4-yl)-ethyl]-5-(4-fluorophenyl)-2-isopropyl-4-phenyl-1H-pyrrole-3-carboxylic acid of Formula VII with aniline is carried out in the presence of p-toluene sulfonic acid.

23. The process of claim 1 wherein the hydrolysis of 6-{2-[2-(4-fluorophenyl)-5-isopropyl-3-phenyl-4-phenylcarbamoyl-pyrrole-1-yl]-ethyl}-2,2-dimethyl-[1,3]dioxan-4-yl)-acetic acid tert-butyl ester of Formula VIII to give (3R,5R)-7-[2-(4-Fluorophenyl)-5-isopropyl-3-phenyl-4-phenylcarbamoyl-pyrrol-1-yl]-3,5-dihydroxy-heptanoic acid of Formula IX is carried out in the presence of a base.

24. The process of claim 23, wherein the base comprises one or more of lithium hydroxide, sodium hydroxide and potassium hydroxide.

25. A pharmaceutical composition comprising an effective amount of atorvastatin or a pharmaceutically acceptable salt thereof obtained by the process of claim 1, and one or more pharmaceutically acceptable carriers, excipients or diluents.

26. A method of treating hypolipidemia, hypocholesterolemia and atherosclerosis in a warm-blooded animal, the method comprising providing a dosage form to the warm-blooded animal that includes atorvastatin or a pharmaceutically acceptable salt thereof obtained by the process of claim 1.
